(19) United States
(12) Patent Application Publication
(10) Pub. No.: US 2018/0340098 A1
(43) Pub. Date: Nov. 29, 2018

(54) ADHESIVE AND ADHESIVE TAPE

(71) Applicants: TOYO INK SC HOLDINGS CO., LTD., Tokyo (JP); TOYOCHEM CO., LTD., Tokyo (JP)

(72) Inventors: Shingo TANABE, Tokyo (JP); Takeshi SATO, Tokyo (JP); Shuhei SAITO, Tokyo (JP)

(73) Assignees: TOYO INK SC HOLDINGS CO., LTD., Tokyo (JP); TOYOCHEM CO., LTD., Tokyo (JP)

(21) Appl. No.: 15/777,914

(22) PCT Filed: Nov. 18, 2016
(86) PCT No.: PCT/JP2016/084273

§ 371 Date: May 22, 2018

(30) Foreign Application Priority Data
Nov. 25, 2015 (JP) 2015-229261

(51) Int. Cl. C09J 7/38; C08F 220/28; C09J 7/28; A61L 15/24; A61L 15/58

(52) U.S. Cl. CPC C09J 7/385; C08F 220/28; C09J 7/28; A61L 15/24; C08F 2800/20; C09J 2205/114; C09J 2433/00; C09J 2400/163; C08F 2220/285; A61L 15/58

(57) ABSTRACT

This adhesive includes a copolymer that includes monomer units represented by (a-1), (a-2), (a-3), and (a-4) described below, and the following content is contained among all of the monomers which constitute the copolymer:
(a-1) 5 to 45 wt % of an alkyl(meth)acrylate;
(a-2) 0.01 to 10 wt % of a (meth)acrylate having a hydroxyl group or an allyl ether having a hydroxyl group;
(a-3) 50 to 78 wt % of an alkoxyalkyl (meth)acrylate;
(a-4) 1 to 25 wt % of an alkoxypolyalkylene glycol mono(meth)acrylate or an alkoxypolyalkylene glycol allyl ether.

### TECHNICAL FIELD

The present invention relates to an adhesive and an adhesive tape. More specifically, the present invention relates to an adhesive and an adhesive tape suitably used for attachment to skin for medical applications.

### BACKGROUND ART

An adhesive tape for attachment to skin generally has a structure in which an adhesive layer containing a drug as necessary is formed on one side of a flexible support. As a function of the adhesive tape for attachment to skin, it is required to prevent invasion of water, bacteria, viruses and the like from the outside into the body, and it is also required to have moisture permeability (also referred to as water vapor permeability) for evaporating moisture due to perspiration from the skin. When moisture permeability is inferior, sweat accumulates between the skin and the adhesive layer so that skin problems such as stuffiness and eruptions are easily caused on the skin, and the adhesive strength is lowered so that the tape easily peels off from the skin. Further, during attachment, the adhesive tape is required to follow the curved surface of the skin or movement such as stretching, twisting and the like so that the adhesive tape hardly peels off. When the adhesive tape is peeled off, it is required that there is little pain and no exfoliation of dead skin cells, and no adhesive remains on the skin.

However, when the adhesive strength is excessively increased to prevent peeling, the hair on the body surface may be removed at the time of peeling off, exfoliation of dead skin cells may occur, or the adhesive may remain on the skin surface. Further, in order to improve the moisture permeability, the copolymerization proportion of the functional group-containing monomer needs to be increased, and as a result, the skin irritation at the time of peeling off may not be relieved.

Patent Literature 1 discloses a medical adhesive having a high water vapor permeability, a moderate adhesive strength to an extent that the adhesive hardly peels off and does not damage the skin when peeled off, and durability of such a moderate adhesive strength and a moderate cohesive strength, and including a polymer containing units derived from an alkoxyalkyl acrylate of which a homopolymer has a glass transition temperature (Tg) of −35° C. or less in an amount of more than 50 wt %. However, medical adhesives having high water vapor permeability have weak adhesive strength, and those having high adhesive strength tend to have low water vapor permeability, and thus there is a problem that the two of water vapor permeability and adhesive strength are incompatible.

Patent Literature 2 discloses an adhesive composition for attachment to skin and an adhesive tape for attachment to skin capable of being used optimally for external application such as in a medical hygiene field, a sports field, a beauty health field and the like, in which the adhesive composition includes a liquid or pasty carboxylic acid ester at room temperature at 20 to 120 parts by weight based on 100 parts by weight of an acrylic copolymer obtained from a monomer mixture including an alkyl (meth)acrylate ester at 40 to 80 wt %, an alkoxy group-containing ethylenic unsaturated monomer at 10 to 60 wt %, and a carboxyl group-containing ethylenic unsaturated monomer at 1 to 10 wt %. However, although it has excellent water resistant adhesion strength for curbing peeling off during perspiration, it has a problem of low moisture permeability.

Patent Literature 3 discloses an adhesive tape capable of being attached well to a dry surface and a wet surface, and having no adhesive residue at the time of peeling off, in which a copolymer including an (meth)acrylic acid alkyl ester, an acrylic acid, and 2-methoxymethyl acrylate is used as an adhesive. However, it focuses attention on attachment to a wet surface, and there is a problem that having both moisture permeability and adhesive strength is not satisfied.

### CITATION LIST

### Patent Literature

[Patent Literature 1] Japanese Unexamined Patent Application Publication No. H7-275340

[Patent Literature 2] Japanese Unexamined Patent Application Publication No. 2002-065841

[Patent Literature 3] Japanese Unexamined Patent Application Publication No. 2006-036911

### SUMMARY OF INVENTION

### Technical Problem

As described above, adhesives having both moisture permeability and adhesive strength are not known, and an adhesive and an adhesive tape capable of satisfying having both of these characteristics are required. Further, in order to use the adhesive for medical applications such as attachment to skin, the adhesive is also required to have good adhesion to the skin, and allow users to be less likely to have a feeling that the tape is attached during attachment of the tape and pain during peeling off of the tape.

Accordingly, an objective to be solved by the present invention is to provide an adhesive and an adhesive tape which exhibit high moisture permeability and excellent adhesive strength. Further, in addition to the above-described objective, another objective of the present invention is to provide an adhesive and an adhesive tape which have excellent adhesion to the skin, and by which users are less likely to have a feeling that the tape is attached during attachment of the tape and pain during peeling off of the tape.

### Solution to Problem

An adhesive of the present invention includes a copolymer at least including monomer units represented by (a-1), (a-2), (a-3), and (a-4) described below, and the following content is contained among all of the monomers which constitute the copolymer:

(a-1) 5 to 45 wt % of an alkyl (meth)acrylate;

(a-2) 0.01 to 10 wt % of a (meth)acrylate having a hydroxyl group or an allyl ether having a hydroxyl group;

(a-3) 50 to 78 wt % of an alkoxyalkyl (meth)acrylate;

(a-4) 1 to 25 wt % of an alkoxypolyalkylene glycol mono(meth)acrylate or an alkoxypolyalkylene glycol allyl ether.

Further, an embodiment of the present invention relates to the adhesive in which a solubility in water at 40° C. is less than 3%.

Further, an embodiment of the present invention relates to the adhesive in which the copolymer has a weight average molecular weight of 800,000 or more and less than 2,000,000.

Further, an embodiment of the present invention relates to the adhesive further including a curing agent.

Further, an embodiment of the present invention relates to the adhesive including a copolymer which at least includes the monomer units represented by (a-1), (a-2), (a-3), and (a-4), and a monomer unit having an amide bond.

Further, an embodiment of the present invention relates to the adhesive in which the copolymer is a copolymer that do not contain monomers having an acidic group.

Further, an embodiment of the present invention relates to the adhesive in which the alkyl (meth)acrylate (a-1) includes an alkyl (meth)acrylate having 4 to 8 carbon atoms at 5 to 40 wt % and an alkyl (meth)acrylate having 9 to 22 carbon atoms at 5 to 40 wt %.

Further, an embodiment of the present invention relates to the adhesive used for medical applications.

Further, an embodiment of the present invention relates to the adhesive in which, after an adhesive tape having an adhesive layer formed of the adhesive is pressure-bonded to a stainless steel plate in an environment of 23° C. and 50% RH, when an adhesive strength measured after 10 minutes in an environment of 23° C. and 50% RH is defined as adhesive strength (1) and an adhesive strength measured after 24 hours in an environment of 40° C. and 50% RH is defined as adhesive strength (2), a ratio of adhesive strength (2)/adhesive strength (1) is less than 3.

Further, an embodiment of the present invention relates to a method of preparing an adhesive, in which at least monomers represented by (a-1), (a-2), (a-3), and (a-4) described below are copolymerized, and which is characterized by copolymerizing the monomers having the following contents among all of the monomers which constitute a copolymer:

(a-1) 5 to 45 wt % of an alkyl (meth)acrylate;

(a-2) 0.01 to 10 wt % of a (meth)acrylate having a hydroxyl group or an allyl ether having a hydroxyl group;

(a-3) 50 to 78 wt % of an alkoxyalkyl (meth)acrylate;

(a-4) 1 to 25 wt % of an alkoxypolyalkylene glycol mono(meth)acrylate or an alkoxypolyalkylene glycol allyl ether.

Further, an embodiment of the present invention relates to an adhesive tape, including an adhesive layer formed of the adhesive.

Further, an embodiment of the present invention relates to the adhesive tape in which, after an adhesive tape having an adhesive layer formed of the adhesive is pressure-bonded to a stainless steel plate in an environment of 23° C. and 50% RH, when an adhesive strength measured after 10 minutes in an environment of 23° C. and 50% RH is defined as adhesive strength (1) and an adhesive strength measured after 24 hours in an environment of 40° C. and 50% RH is defined as adhesive strength (2), a ratio of adhesive strength (2)/adhesive strength (1) is less than 3.

Further, an embodiment of the present invention relates to a method of preparing an adhesive tape, in which the adhesive is applied onto a substrate.

### Advantageous Effects of Invention

When used for attachment to skin, an adhesive and adhesive tape obtained by the present invention have high moisture permeability, excellent adhesive strength and durability of attachment to skin, and hardly cause skin problems such as stuffiness and eruptions. Further, there is little pain at the time of peeling off from the skin, no exfoliation of dead skin cells, and no adhesive remaining on the skin. Moreover, a sticky feeling without an uncomfortable feeling can be obtained since the adhesive tape follows the movement of the skin when the adhesive tape is attached thereto.

### DESCRIPTION OF EMBODIMENTS

Hereinafter, the details of the present invention will be described. In the present specification, unless otherwise specified, the terms “(meth)acryl”, “(meth)acryloyl”, “(meth)acrylic acid”, “(meth)acrylate”, “(meth)acryloyloxy” and “(meth) allyl” refer to “acryl or methacryl”, “acryloyl or methacryloyl”, “acrylic acid or methacrylic acid”, “acrylate or methacrylate”, “acryloyloxy or methacryloyloxy”, and “ally! or methallyl”, respectively.

An acrylic adhesive of the present invention is an adhesive including a copolymer which at least includes monomer units represented by (a-1), (a-2), (a-3), and (a-4) described below:

(a-1) an alkyl (meth)acrylate;

(a-2) a (meth)acrylate having a hydroxyl group or an allyl ether having a hydroxyl group;

(a-3) an alkoxyalkyl (meth)acrylate;

(a-4) an alkoxypolyalkylene glycol mono(meth)acrylate or an alkoxypolyalkylene glycol allyl ether.

<(a-1) Alkyl(meth)acrylate>

Examples of the alkyl (meth)acrylate which is a monomer represented by (a-1) include alkyl (meth)acrylates such as methyl (meth)acrylate, ethyl (meth)acrylate, butyl (meth)acrylate, 2-ethylhexyl (meth)acrylate, nonyl (meth)acrylate, decyl (meth)acrylate, lauryl (meth)acrylate, cetyl (meth)acrylate, stearyl (meth)acrylate, behenyl (meth)acrylate and the like.

Among these alkyl (meth)acrylates, an alkyl (meth)acrylate having an alkyl group having 4 to 22 carbon atoms is preferable, an alkyl (meth)acrylate having an alkyl group having 4 to 16 carbon atoms is more preferable, and an alkyl (meth)acrylate having an alkyl group having 8 to 14 carbon atoms is still more preferable.

Adhesive tapes for attachment to skin may become wet with water, and have a certain level of hydrophobicity to prevent entry of water from the interface between the adhesive tape and the skin and to prevent the tape from peeling off from the skin. By using an alkyl (meth)acrylate, hydrophobicity is strengthened, and durability of attachment to skin is improved. In particular, adhesiveness and hydrophobicity can be imparted and durability of attachment to skin is increased by one type or a combination of two or more types of an alkyl (meth)acrylate having 4 to 22 carbon atoms. For adjusting the hydrophobicity, it is preferable to use two or more types of an alkyl (meth)acrylate having 4 to 22 carbon atoms.

The content of the alkyl (meth)acrylate is preferably in the range of 5 to 45 wt %, more preferably 5 to 40 wt %, and still more preferably 10 to 40 wt %, in all of the monomers constituting the copolymer. When the content is 45 wt % or less, the adhesive strength is not excessively increased, and good moisture permeability is achieved due to the amount of monomers contributing to moisture permeability being secured. On the other hand, when the content is 5 wt % or more, adhesiveness to the skin may be secured, and durability of attachment to skin is also developed.

When two or more types of an alkyl (meth)acrylate having 4 to 22 carbon atoms are used, it is preferable that the content of the alkyl (meth)acrylate having 4 to 8 carbon atoms is in the range of 5 to 40 wt %, preferably 10 to 35%, and the content of the alkyl (meth)acrylate having 9 to 22 carbon atoms is in the range of 5 to 40 wt %, preferably 5 to 30 wt %.

<(a-2) (Meth)acrylate Having Hydroxyl Group or Allyl Ether Having Hydroxyl Group>

Examples of a (meth)acrylate having a hydroxyl group or an allyl ether having a hydroxyl group which is a monomer represented by (a-2) include a (meth)acrylate having a hydroxyl group such as 2-hydroxyethyl (meth)acrylate, 3-hydroxypropyl (meth)acrylate, 3-chloro-2-hydroxypropyl (meth)acrylate, 4-hydroxybutyl (meth)acrylate, 6-hydroxyhexyl (meth)acrylate, 8-hydroxyoctyl (meth)acrylate, a polyalkylene glycol mono (meth)acrylate and the like; and an allyl ether having a hydroxyl group such as a polyalkylene glycol allyl ether and the like. Among them, 2-hydroxyethyl (meth)acrylate and 4-hydroxybutyl (meth)acrylate are preferable.

The (meth)acrylate having a hydroxyl group or the allyl ether having a hydroxyl group is preferably contained in an amount of 0.01 to 10 wt %, more preferably 0.1 to 10 wt %, and still more preferably 0.1 to 5 wt % in all of the monomers constituting the copolymer. When the content of the (meth)acrylate having a hydroxyl group or the allyl ether having a hydroxyl group is 0.01 wt % or more, a sufficiently crosslinked structure may be formed with a curing agent, and the adhesive hardly remains on the skin. On the other hand, when the content is 10 wt % or less, an excessively crosslinked structure may be avoided and adhesiveness to the skin may be secured.

<(a-3) Alkoxyalkyl (meth)acrylate>

An alkoxyalkyl (meth acrylate) which is a monomer represented by (a-3) is not particularly limited as long as it is a (meth)acrylate having an alkoxyalkyl group bonded thereto as an alcohol-derived moiety of a (meth)acrylate ester structure.

As the alkoxyalkyl (meth)acrylate, an alkoxyalkyl (meth)acrylate in which an alkoxy group has 1 to 12 carbon atoms and an alkylene group bonded to the alkoxy group has 1 to 18 carbon atoms is preferable. An alkoxyalkyl (meth)acrylate in which an alkoxy group has 1 to 4 carbon atoms and an alkylene group bonded to the alkoxy group has 1 to 8 carbon atoms is more preferable, and an alkoxyalkyl (meth)acrylate in which an alkoxy group has 1 to 2 carbon atoms and an alkylene group bonded to the alkoxy group has 1 to 4 carbon atoms is still more preferable.

Examples of the alkoxyalkyl (meth)acrylate include 2-methoxymethyl (meth)acrylate, 2-methoxyethyl (meth)acrylate, 2-ethoxymethyl (meth)acrylate, 2-ethoxyethyl (meth)acrylate, 3-methoxypropyl (meth)acrylate, 3-ethoxypropyl (meth)acrylate, 4-methoxybutyl (meth)acrylate, 4-ethoxybutyl (meth)acrylate and the like. Among them, 2-methoxyethyl (meth)acrylate and 2-ethoxyethyl (meth)acrylate are particularly preferable.

Since an alkoxy group has high hydrophilicity, it is thought to be excellent in holding and release of water vapor. In order to obtain sufficient moisture permeability, the content of the alkoxyalkyl (meth)acrylate is preferably in the range of 50 to 78 wt %, more preferably 50 to 75 wt %, and still more preferably 55 to 75 wt % in all of the monomers constituting the copolymer. When the content is 50 wt % or more, sufficient moisture permeability is obtained, and when the content is 80 wt % or less, sufficient adhesiveness to the skin is developed.

Since the monomer (a-1) has high hydrophobicity and the monomer (a-4) which will be described below has high hydrophilicity, these are not compatible with each other and a copolymer formed of only the monomer (a-1) and the monomer (a-4) is likely to undergo phase separation and may cause deterioration of adhesiveness and storage stability. It is thought that, by using the monomer (a-3) as a monomer constituting the copolymer, the compatibility between the monomer (a-1) and the monomer (a-4) is improved, and adhesiveness and storage stability are improved.

<(a-4) Alkoxypolyalkylene Glycol Mono(meth)acrylate or an Alkoxypolyalkylene Glycol Allyl Ether>

An alkoxypolyalkylene glycol mono (meth)acrylate is a compound represented by the following Formula (1).

CH₂═CR1-CO—(OZ)ₙ—O—R2 Formula (1)

(In Formula (1), Z represents an alkylene group, R1 represents hydrogen or a methyl group, R2 represents an alkyl group, and n represents an integer of 2 or more.)

In Formula (1), it is preferable that Z is an alkylene group having 2 to 3 carbon atoms, R2 is an alkyl group having 1 to 2 carbon atoms, and n is an integer of 2 to 20.

Examples of the alkoxypolyalkylene glycol mono(meth)acrylate include MPE400A, MPE550A (all of which are manufactured by Osaka Organic Chemical Industry Ltd.), Light Ester BC, 130MA, 041MA, Light Acrylate EC-A, MTG-A, EHDG-AT 130A, DPM-A (all of which are manufactured by Kyoeisha Chemical Co., Ltd.), FANCRYL FA-400M (100) (manufactured by Hitachi Chemical Co., Ltd.), NK Ester AM-90, AM-130G, M-90G, M-230G (all of which are manufactured by Shin-Nakamura Chemical Co., Ltd.), BLEMMER AME-400, PME-100, PME-200, PME-400, PME-1000 (all of which are manufactured by NOF Corporation), and the like.

An alkoxypolyalkylene glycol allyl ether is a compound represented by the following Formula (2).

CH₂═CR3-CH₂—O—(YO)ₘ—R4 Formula (2)

(In Formula (2), Y represents an alkylene group, R3 represents hydrogen or a methyl group, R4 represents an alkyl group, and m represents an integer of 1 or more.)

In Formula (2), it is preferable that Y is an alkylene group having 2 to 4 carbon atoms, R4 is an alkyl group having 1 to 4 carbon atoms, m is preferably an integer of 1 to 30, and more preferably, Y is an alkylene group having 2 to 3 carbon atoms, R4 is an alkyl group having 1 to 2 carbon atoms, and m is an integer of 1 to 20.

Examples of the alkoxypolyalkylene glycol allyl ether include UNIOX PKA-5006, PKA-5009, UNSYPE PKA-5015 (all manufactured by NOF Corporation), and the like.

The content of the alkoxypolyalkylene glycol mono(meth)acrylate or an alkoxypolyalkylene glycol allyl ether is preferably in the range of 1 to 25 wt %, and more preferably 5 to 25 wt % in all of the monomers constituting the copolymer. By including the alkylene glycol moiety, it is thought that an excellent performance for holding and releasing water vapor is exhibited. Among the above, an alkoxypolyalkylene glycol mono (meth)acrylate is preferable.

The monomer (a-3) and the monomer (a-4) are components contributing to moisture permeability. The total content of the monomer (a-3) and the monomer (a-4) in all the monomers constituting the copolymer is preferably in the range of 50 to 80 wt %. When the content is 50 wt % or more, sufficient moisture permeability is obtained, and when the content is 80 wt % or less, adhesion durability to the skin may be secured.

The copolymer preferably contains a monomer unit having an amide bond as a monomer constituting the copolymer.

Examples of the monomer having an amide bond include (meth)acrylamide, N-methyl (meth)acrylamide, N-ethyl (meth)acrylamide, N-propyl (meth)acrylamide, N-hexyl (meth)acrylamide, N-vinyl pyrrolidone, acryloyl morpholine, vinyl caprolactam and the like.

When a monomer having an amide bond is used, the content thereof is in the range of 0.05 to 5 wt %, preferably 0.1 to 4% wt %, and more preferably 0.1 to 3 wt %, in all of the monomers constituting the copolymer. The monomer having an amide bond acts as a reaction catalyst between a hydroxyl group in the copolymer and an isocyanate compound of a curing agent which will be described below, and contributes to the stabilization of the reaction.

The copolymer may contain other monomers than the above-described monomers as a monomer constituting the copolymer.

Examples of other monomers include alicyclic group-containing monomers such as cyclohexyl (meth)acrylate, isobornyl (meth)acrylate and the like; aromatic group-containing monomers such as benzyl (meth)acrylate, phenoxyethyl (meth)acrylate and the like; amino group-containing monomers such as dimethylaminoethyl (meth)acrylate, diethylaminoethyl (meth)acrylate and the like; a cyano group-containing monomer such as cyano (meth)acrylate and the like; maleimide group-containing monomers such as maleimide, methyl maleimide and the like; a nitrile group-containing monomer such as (meth)acrylonitrile and the like; an epoxy group-containing monomer such as glycidyl (meth)acrylate and the like; and a vinyl ester such as vinyl acetate and the like.

The other monomers may be copolymerized in an amount of 0 to 20 wt %, preferably 0 to 15 wt %, and more preferably 0 to 10 wt %, in all of the monomers constituting the copolymer.

The copolymer may contain a monomer having an acidic group as the other monomers. When a monomer having an acidic group is used, there is an advantage that the adhesive strength may be increased and the adhesion durability may be improved, but on the other hand, there is a tendency that strong pain or exfoliation of dead skin cells occurs at the time of peeling off the tape. The adhesive of the present invention preferably does not contain a monomer having an acidic group.

Examples of the monomer having an acidic group include (meth)acrylic acid, maleic acid, itaconic acid, a monomer having a phosphoric acid group, a monomer having a sulfuric acid group and the like.

The acrylic copolymer of the present invention preferably has a weight average molecular weight of 500,000 or more and less than 2,000,000, more preferably 500,000 or more and less than 1,500,000. When the weight average molecular weight is 500,000 or more, the adhesive hardly remains on the skin, and when the weight average molecular weight is less than 2,000,000, skin attachment properties become excellent.

The copolymer preferably has a glass transition temperature (Tg) determined by Fox's equation in the range of −70° C. to 0° C. By using a (meth)acrylic copolymer having such a glass transition temperature, it is possible to obtain an adhesive which is excellent in durability of attachment to skin and prevents pain during peeling.

The copolymer may be prepared by a known method, but it is preferably prepared by solution polymerization. In solution polymerization, it is preferable to use methyl acetate, ethyl acetate, methyl ethyl ketone, toluene, acetone or the like as a solvent.

Specifically, a solvent and the raw material monomers are charged into a reaction vessel in the proportions shown above, a polymerization initiator is added under an inert gas atmosphere such as nitrogen gas, and the reaction temperature is increased to about 50 to 90° C. to perform a polymerization reaction for 4 to 20 hours.

Examples of the polymerization initiator include an azo-type initiator and a peroxide-type initiator. These polymerization initiators are used usually in an amount within the range of 0.01 to 5 parts by weight with respect to 100 parts by weight of the raw material monomers. Further, during the polymerization reaction, a chain transfer agent, raw material monomers, and a solvent may be appropriately added.

Under the above-mentioned conditions, the weight average molecular weight of the obtained copolymer may be adjusted according to known techniques by adjusting reaction conditions such as the type and amount of a solvent to be used, the type and amount of polymerization initiator, the reaction time, the reaction temperature and the like.

<Curing Agent>

As a curing agent for curing the copolymer, it is preferable to use a compound having an isocyanate group (hereinafter, referred to as an isocyanate-based curing agent). Examples of the isocyanate-based curing agent include compounds having two or more isocyanate groups in the molecule such as tolylene diisocyanate, xylylene diisocyanate, chlorophenylene diisocyanate, hexamethylene diisocyanate, tetramethylene diisocyanate, isophorone diisocyanate, hydrogenated diphenylmethane diisocyanate and the like; compounds obtained by subjecting these to an addition reaction with polyhydric alcohols such as trimethylolpropane and pentaerythritol, isocyanurate compounds thereof and burette type compounds thereof, and a urethane prepolymer-type compound having two or more isocyanate groups in the molecule obtained by subjecting these to an addition reaction with a known polyether polyol, polyester polyol, acryl polyol, polybutadiene polyol, polyisoprene and the like.

The curing agent may be appropriately selected depending on the substrate, application, etc. Tolylene diisocyanate, xylylene diisocyanate, hexamethylene diisocyanate, isophorone diisocyanate; compounds obtained by subjecting these to an addition reaction with polyalcohols such as trimethylolpropane, pentaerythritol and the like, and isocyanurate compounds thereof and burette type compounds thereof may be preferably used.

The proportion of the curing agent mixed in is preferably in the range of 0.01 to 5 parts by weight, more preferably 0.1 to 3 parts by weight, and still more preferably 0.1 to 2 parts by weight based on 100 parts by weight of the copolymer, although it depends on the amount of the functional groups contained in the copolymer.

Various additives may be added to the adhesive of the present invention, as desired. For example, antibacterial agents, humectants, vitamins, perfumes and the like may be mentioned. These are mixed in in an effective amount as necessary.

<Adhesive Tape>

The adhesive tape prepared using the adhesive of the present invention may be sterilized with an autoclave, ethylene oxide gas, radiation, etc. In order to prevent deterioration of the adhesive due to radiation sterilization, known additives may be used. An electron/ion scavenger which inactivates electrons and ions generated in the early stage of radiation irradiation such as pyrene, quinone and the like, an energy transfer agent which inactivates excited species such as acenaphthene, a radical scavenger which inactivates polymer radicals such as mercaptan, octahydrophenanthrene, a monoalkyl diphenyl ether and the like, an antioxidant which inactivates polymer radicals and peroxy radicals such as a phenolic antioxidant, an organic thioacid salt, phosphorous acid triester, BHT and the like, a plasticizer which inactivates polymer radicals such as a phthalic acid ester, an epoxy plasticizer, a chlorinated paraffin and the like may be mentioned.

In order to prepare an adhesive tape for attachment to skin using the adhesive of the present invention, the adhesive is applied on one surface of a substrate. The substrate is not particularly limited, but main examples thereof include a woven fabric, a nonwoven fabric and a knitted fabric in which a single substance of rayon, cotton, polyester yarn and the like or polyurethane yarn is inserted, and a film or a sheet formed of a synthetic resin such as plasticized flexible polyvinyl chloride, non-plasticized flexible polyvinyl chloride, polyethylene, polypropylene, polybutene, an ethylene-methyl methacrylate copolymer, an ethylene-vinyl acetate copolymer, ethylene-propylene rubber, polyamide, polyethylene terephthalate, polyurethane, polyvinylidene chloride, polyvinyl alcohol, etc. Further, a flexible film or sheet such as a plastic foam sheet, cellulose, cellulose acetate or the like may be used. Corona discharge treatment or the like may be performed on these base materials as necessary. When a base material having excellent moisture permeability is used, the excellent moisture permeability of the adhesive of the present invention may be sufficiently exhibited

The adhesive of the present invention may be diluted with a solvent so as to facilitate coating. As the solvent, known solvents may be used, and examples thereof include ethyl acetate, acetone, methyl ethyl ketone, toluene and the like. An adhesive solution is mixed with a curing agent or an additive, and a mixture is applied on one surface of the substrate and dried. In the case where high temperature is required for the crosslinking reaction, heating is performed during drying or after drying. Further, it is also possible to adopt a method in which an adhesive composition is applied to a release liner such as a release paper and then the adhesive composition is layered on a substrate. When perforation is provided in the adhesive tape, air permeability may be improved.

The acrylic adhesive of the present invention is suitable for attachment to skin, and has a chance of getting wet in a case such as when users wash their hands or have a bath, and thus preferably has a composition which is hardly soluble in water. To this end, the acrylic adhesive of the present invention preferably has a solubility in water at 40° C. of less than 3 wt %, more preferably, 2 wt % or less, and still more preferably, 1 wt % or less. The lower the solubility in water, the greater the stability of adhesion to skin wet with water or underwater.

Hereinafter, the present invention will be specifically described with reference to examples and comparative examples. In the following examples, “parts” and “%” represent “parts by mass” and “mass %”, respectively, unless otherwise specified. Further, the abbreviations of the materials used in the following examples are as below.

<(a-1) Alkyl (meth)acrylate>

BA: n-butyl acrylate

2EHA: 2-ethylhexyl acrylate

IOA: isooctyl acrylate

INA: isononyl acrylate

IDMA: isodecyl methacrylate

LA: lauryl acrylate

LMA: lauryl methacrylate

ISA: isostearyl acrylate

<(a-2) (Meth)acrylate Having a Hydroxyl Group or an Allyl Ether Having a Hydroxyl Group>

2HEA: 2-hydroxyethyl acrylate

4HBA: 4-hydroxybutyl acrylate

55PET-800: Blemmer 55 PET-800, polyethylene glycol-tetramethylene glycol-monomethacrylate (random type), manufactured by NOF Corporation

PKA-5001: UNIOX PKA-5001, polyethylene glycol-allyl ether, manufactured by NOF Corporation

<(a-3) Alkoxyalkyl (meth)acrylate>

MEA: methoxyethyl acrylate

MEMA: methoxyethyl methacrylate

EEA: ethoxyethyl acrylate

<(a-4) Alkoxypolyalkylene Glycol Mono(meth)acrylate or an Alkoxypolyalkylene Glycol Allyl Ether>

EC-A: light acrylate EC-A, ethoxy-diethylene glycol acrylate, manufactured by Kyoeisha Chemical Co., Ltd.

MTG-A: light acrylate MTG-A, methoxy-triethylene glycol acrylate, manufactured by Kyoeisha Chemical Co., Ltd.

DPM-A: light acrylate DPM-A, methoxydipropylene glycol acrylate, manufactured by Kyoeisha Chemical Co., Ltd.

M-90G: NK Ester M-90G, methoxypolyethylene glycol methacrylate, manufactured by Shin-Nakamura Chemical Co., Ltd.

AM-90G: NK Ester AM-90G, methoxypolyethylene glycol acrylate, manufactured by Shin-Nakamura Chemical Co., Ltd.

MPE 400A: methoxypolyethylene glycol acrylate, manufactured by Osaka Organic Chemical Industry Ltd.

BC: light ester BC, butoxydiethylene glycol methacrylate, manufactured by Kyoeisha Chemical Co., Ltd.

041 MA: light ester 041MA, methoxy polyethylene glycol methacrylate, manufactured by Kyoeisha Chemical Co., Ltd.

PKA-5008: Uniox PKA-5008, methoxy-polyethylene glycol-allyl ether, manufactured by NOF Corporation

PKA-5015: Unisafe PKA-5015, butoxy-polyethylene glycol-polypropylene glycol-allyl ether, manufactured by NOF Corporation

<Other Monomers>

AA: acrylic acid

AM: acrylamide

NVP: N-vinylpyrrolidone

ACMO: acryloyl morpholine

<Curing Agent>

HT: Sumidur HT, hexamethylene diisocyanate-trimethylolpropane form, manufactured by Covestro Japan Ltd.

N 3200: Desmodur N 3200, hexamethylene diisocyanate-burette form, manufactured by Covestro Japan Ltd.

N3300: Desmodur N3300, hexamethylene diisocyanate-nurate form, manufactured by Covestro Japan Ltd.

L: Coronate L, tolylene diisocyanate-trimethylolpropane form, manufactured by Tosoh Corporation

2030: Coronate 2030, tolylene diisocyanate-nurate form, manufactured by Tosoh Corporation

D-110N: Takenate D-110N, xylylene diisocyanate-trimethylolpropane form, manufactured by Mitsui Chemicals, Inc.

EX-212: Denacol EX-212, 1,6-hexanediol diglycidyl ether, manufactured by Nagase ChemteX Corporation

### EXAMPLES

### Example 1

26 parts of 2-ethylhexyl acrylate, 2.5 parts of 2-hydroxyethyl acrylate, 20 parts of methoxyethyl acrylate, 1.5 parts of methoxypolyethylene glycol methacrylate (Light Ester 041MA, manufactured by Kyoeisha Chemical Co., Ltd.), 69 parts of ethyl acetate and 0.2 parts of azobisisobutyronitrile were placed in a four-necked flask equipped with a stirrer, a reflux condenser, a nitrogen inlet tube, a thermometer and a dropping funnel, and heated to 80° C. while nitrogen gas was introduced. 26 parts of 2-ethylhexyl acrylate, 2.5 parts of 2-hydroxyethyl acrylate, 20 parts of methoxyethyl acrylate, 1.5 parts of methoxypolyethylene glycol methacrylate (Light Ester 041MA, manufactured by Kyoeisha Chemical Co., Ltd.), 69 parts of ethyl acetate and 0.2 parts of azobisisobutyronitrile as a polymerization initiator were charged into a dropping funnel and this mixture was added dropwise, and a polymerization reaction was carried out at 80° C. under a nitrogen atmosphere for 7 hours. After completion of the reaction, dilution was performed by adding 48 parts of ethyl acetate to obtain an adhesive having a nonvolatile content of 35% and a weight average molecular weight of 800,000.

<Weight Average Molecular Weight (Mw)>

Mw was measured under the following conditions. The determination of Mw was performed by a calibration curve method using polystyrene having a known weight average molecular weight as a standard substance.

Device name: LC-GPC system “Prominence” manufactured by Shimadzu Corporation

Column: TSKgel α-M manufactured by Tosoh Corporation: two connected in series.

Mobile phase solvent: tetrahydrofuran

Flow rate: 1.0 ml/min

Column temperature: 40° C.

<Solubility>

Approximately 600 mg of the nonvolatile content of the adhesive was accurately weighed out, placed in a stoppered flask and 300 mL of ultrapure water having a pH of 7 was added thereto. The mixture was stirred at 35 to 40° C. for 60 minutes, then placed in a constant temperature bath at 40° C. and gently stirred for 24 hours. The residue which remained undissolved was dried under vacuum and the mass was measured. The solubility was determined based on the following Equation (3). Evaluation standards were as follows.

Solubility (%)={1−(mass of residue/mass of nonvolatile content before dissolution)}×100 (3)

◯: Solubility was less than 1% (very good)

Δ: Solubility was 1% or more (good)

×: Solubility was 3% or more (poor)

<Preparation of Coating Solution>

With respect to 100 parts of the nonvolatile content of the obtained acrylic adhesive, 2 parts of Sumidur HT as a curing agent was added in terms of nonvolatile content, and a mixture was stirred to obtain a coating solution.

<Preparation of Adhesive Tape>

An adhesive tape was prepared according to the method as described below and the properties were evaluated.

(1) Preparation of Adhesive Tape for Adhesive Strength Evaluation

A polyester film (trade name “E5100”, manufactured by Toyobo Co., Ltd., hereinafter the same) having a thickness of 25 μm was coated with the coating solution obtained as above so that a thickness after drying was 25 μm, and dried in a hot air oven at 100° C. for 2 minutes. After drying, a separator formed of polyester (trade name “Superstick” SP-PET 38, manufactured by Lintec Corporation, hereinafter the same) having a thickness of 38 μm was laminated thereon, and further cured at 50° C. for 3 days to obtain an adhesive tape.

(2) Preparation of Adhesive Tape for Evaluating Moisture Permeability, Adhesion to Skin, Irritation to Skin-1 and Irritation to Skin-2

The above-described coating solution was applied to a polyester separator having a thickness of 38 μm so that the thickness after drying was 25 μm, and dried in a hot air oven at 100° C. for 2 minutes. After drying, a moisture-permeable urethane sheet (moisture permeability: 3,500 g/m²·24 h) having a thickness of 38 μm was laminated thereon and further cured at 50° C. for 3 days to obtain an adhesive tape.

<Moisture Permeability>

A test according to JIS L 1099 was conducted. 42 g of pure water at 40° C. was placed in a cylindrical weighing container having a diameter of 70 mm, and the container was sealed by fixing the adhesive tape as the cover of the weighing container so that the adhesive-coated surface of the adhesive tape faced downward. The container was allowed to stand for 1 hour in a constant temperature and constant humidity chamber at 40° C. and a relative humidity of 50% RH to calculate the permeated water content per 1 square meter area for about 24 hours from the weight loss of the weighing container as the moisture permeability (unit: g/m²·24 h) of the adhesive tape.

⊚: 2000 g/m²·24 h or more (very good)

◯: 1500 g/m²·24 h or more, less than 2000 g/m²·24 h (good)

Δ: 1000 g/m²·24 h or more, less than 1500 g/m²·24 h (usable)

×: Less than 1000 g/m²·24 h (poor)

<Adhesive Strength>

The adhesive tape was cut into a size of 25 mm in width and 100 mm in length to obtain a measurement sample. Subsequently, a separator formed of polyester was peeled off from the sample in an environment of 23° C. and 50% RH, and an exposed adhesive layer was adhered to a stainless steel plate (SUS304) and pressure-bonded by a 2 kg roll. After 10 minutes of pressure-bonding, the adhesive strength was measured under the conditions of a peel rate of 300 mm/min and a peeling angle of 180° in accordance with JIS Z 0237, using a tensile tester (this adhesive strength is referred to as “adhesive strength (1)”). After standing for 60 minutes after pressure-bonding, the plate was allowed to stand in an environment of 40° C. and 50% RH for 24 hours, and then left in a 23° C. and 50% RH environment for 60 minutes, and the adhesive strength was similarly measured (this adhesive strength is referred to as “adhesive strength (2)”). Evaluation standards were as follows.

◯: 5 N/25 mm or more, less than 20 N/25 mm (good)

×: less than 5 N/25 mm (poor)

Further, the ratio of adhesive strength (2)/adhesive strength (1) (may be referred to as an adhesive strength ratio) was calculated and evaluated according to the following criteria.

◯: Less than 2 (very good)

Δ: 2 or more but less than 3 (good)

×: 3 or more (poor)

<Adhesion to Skin>

Adhesion to skin after 24 hours passed after adhering an adhesive tape of 20 mm×80 mm to an adherend (front part of the forearm of a volunteer person, hereinafter the same) was evaluated according to the following criteria.

Evaluation criteria:

◯: Wholly adhered and peeling was not observed (very good)

Δ: Partial peeling was observed (good)

×: Completely peeled off (poor)

<Irritation to Skin-1>

An adhesive tape of 20 mm×80 mm was adhered to the adherend, and the sticking feeling was evaluated according to the following criteria.

◯: Skin was not pulled (very good)

Δ: There was a feeling that the skin was slightly pulled, but deformation was not observed in the skin around the adhered face (good)

×: There was a feeling that the skin was strongly pulled, deformation was observed in the skin around the adhered face (poor)

<Irritation to Skin-2>

An adhesive tape of 20 mm×80 mm was adhered to the adherend and the skin irritation at the time of peeling off the adhesive tape after 24 hours passed was evaluated according to the following criteria.

◯: Pain was not felt (good)

Δ: Although slight pain was felt, erythema was not observed on the peeled surface (usable)

×: Strong pain was felt, erythema was found on the peeled surface (poor)

### Examples 2 to 10 and 12 to 28

Adhesives and adhesive tapes respectively were prepared in the same manner as in Example 1 except that the materials, compositions and amounts were changed as shown in Table 1, and measurement and evaluation were performed.

### Example 11

4.8 parts of 2-ethylhexyl acrylate, 9 parts of lauryl methacrylate, 0.5 parts of 4-hydroxybutyl acrylate, 27.5 parts of ethoxyethyl acrylate, 7.5 parts of ethoxydiethylene glycol acrylate (Light Acrylate EC-A: manufactured by Kyoeisha Chemical Co., Ltd.), 0.8 parts of N-vinylpyrrolidone, 25 parts of ethyl acetate, 50 parts of methyl ethyl ketone, and 0.2 parts of azobisisobutyronitrile were charged into a four-necked flask equipped with a stirrer, a reflux condenser, a nitrogen inlet tube, a thermometer and a dropping funnel, and the temperature was raised to 80° C. while nitrogen gas was introduced. 4.8 parts of 2-ethylhexyl acrylate, 9 parts of lauryl methacrylate, 0.5 parts of 4-hydroxybutyl acrylate, 27.5 parts of ethoxyethyl acrylate, 7.5 parts of ethoxydiethylene glycol acrylate (Light Acrylate EC-A: manufactured by Kyoeisha Chemical Co., Ltd.), 0.8 parts of N-vinylpyrrolidone and 75 parts of ethyl acetate, and 0.2 parts of azobisisobutyronitrile as an initiator were charged into the dropping funnel, the mixture was added dropwise, and the polymerization reaction was carried out at 80° C. under a nitrogen atmosphere for 7 hours. After completion of the reaction, dilution was performed by adding 36 parts of ethyl acetate to obtain an adhesive having a nonvolatile content of 35% and a weight average molecular weight of 400,000.

Further, adhesives and adhesive tapes each were prepared in the same manner as in Example 1 except that the type of curing agent was changed as shown in Table 1, and measurement and evaluation were performed.

### Comparative Example 1

41.5 parts of 2-ethylhexyl acrylate, 5 parts of isostearyl acrylate, 2.5 parts of 2-hydroxyethyl acrylate, 1.0 part of N-vinyl pyrrolidone, 50 parts of ethyl acetate as a polymerization solvent, and 0.2 parts of azobisisobutyronitrile were charged into a four-necked flask equipped with a stirrer, a reflux condenser, a nitrogen inlet tube, a thermometer and a dropping funnel, and the temperature was raised to 80° C. while nitrogen gas was introduced. 41.5 parts of 2-ethylhexyl acrylate, 5 parts of isostearyl acrylate, 2.5 parts of 2-hydroxyethyl acrylate, 1.0 part of N-vinyl pyrrolidone and 50 parts of ethyl acetate, and 0.2 parts of azobisisobutyronitrile as an initiator were charged into the dropping funnel, the mixture was added dropwise, and the polymerization reaction was carried out at 80° C. under a nitrogen atmosphere for 7 hours. After completion of the reaction, dilution was performed by adding 86 parts of ethyl acetate to obtain an adhesive having a nonvolatile content of 35% and a weight average molecular weight of 600,000.

Further, adhesives and adhesive tapes each were prepared in the same manner as in Example 1 except that the type of curing agent was changed as shown in Table 1, and measurement and evaluation were performed.

<Preparation of Coating Solution>

With respect to 100 parts of the nonvolatile content of the obtained acrylic adhesive, 1.0 part by weight of Coronate L as a curing agent was added in terms of nonvolatile content, and a mixture was stirred to obtain a coating solution. An adhesive tape was obtained in the same manner as in Example 1.

### Comparative Example 2

18.5 parts of isooctyl acrylate, 30 parts of methoxypolyethylene glycol acrylate (NK Ester AM-90G: manufactured by Shin-Nakamura Chemical Co., Ltd.), 1.5 parts of acryloyl morpholine, 30 parts of ethyl acetate as a polymerization solvent, 20 parts of methyl ethyl ketone and 0.2 parts of azobisisobutyronitrile were charged into a four-necked flask equipped with a stirrer, a reflux condenser, a nitrogen inlet tube, a thermometer and a dropping funnel, and the temperature was raised to 80° C. while nitrogen gas was introduced. 18.5 parts of isooctyl acrylate, 30 parts of methoxypolyethylene glycol acrylate (NK Ester AM-90G: manufactured by Shin-Nakamura Chemical Co., Ltd.), 1.5 parts of acryloyl morpholine, 50 parts of ethyl acetate and 0.2 parts of azobisisobutyronitrile as an initiator were charged into the dropping funnel, the mixture was added dropwise, and the polymerization reaction was carried out at 80° C. under a nitrogen atmosphere for 7 hours. After completion of the reaction, dilution was performed by adding 86 parts of ethyl acetate to obtain an adhesive having a nonvolatile content of 35% and a weight average molecular weight of 800,000.

<Preparation of Coating Solution>

An adhesive tape was obtained in the same manner as in Example 1 except that no curing agent was added to the obtained acrylic adhesive to prepare a coating solution.

### Comparative Example 3

32.5 parts of isononyl acrylate, 15 parts of methoxyethyl acrylate, 2.5 parts of acrylic acid, 50 parts of ethyl acetate as a polymerization solvent and 0.2 parts of azobisisobutyronitrile were charged into a four-necked flask equipped with a stirrer, a reflux condenser, a nitrogen inlet tube, a thermometer and a dropping funnel, and the temperature was raised while nitrogen gas was introduced. 32.5 parts of isononyl acrylate, 15 parts of methoxyethyl acrylate, 2.5 parts of acrylic acid, 50 parts of ethyl acetate as a polymerization solvent and 0.2 parts of azobisisobutyronitrile were charged into the dropping funnel, the mixture was added dropwise, and the polymerization reaction was carried out at 80° C. under a nitrogen atmosphere for 7 hours. After completion of the reaction, dilution was performed by adding 86 parts of ethyl acetate to obtain an adhesive having a nonvolatile content of 35% and a weight average molecular weight of 800,000.

<Preparation of Coating Solution>

With respect to 100 parts of the nonvolatile content of the obtained acrylic adhesive, Sumidur HT as a curing agent was added in an amount of 0.1 parts by weight in terms of nonvolatile content, and the mixture was stirred to obtain a coating solution. An adhesive tape was obtained in the same manner as in Example 1.

### Comparative Example 4

26.5 parts of 2-ethylhexyl acrylate, 22.5 parts of methoxyethyl acrylate, 1.0 part of acrylic acid, 50 parts of ethyl acetate as a polymerization solvent, and 0.2 parts of azobisisobutyronitrile were charged into a four-necked flask equipped with a stirrer, a reflux condenser, a nitrogen inlet tube, a thermometer and a dropping funnel, and the temperature was raised while nitrogen gas was introduced. 26.5 parts of 2-ethylhexyl acrylate, 22.5 parts of methoxyethyl acrylate, 1.0 part of acrylic acid and 50 parts of ethyl acetate, and 0.2 parts of azobisisobutyronitrile as an initiator were charged into the dropping funnel, the mixture was added dropwise, and the polymerization reaction was carried out at 80° C. under a nitrogen atmosphere for 7 hours. After completion of the reaction, dilution was performed by adding 86 parts of ethyl acetate to obtain an adhesive having a nonvolatile content of 35% and a weight average molecular weight of 900,000.

<Preparation of Coating Solution>

With respect to 100 parts of the nonvolatile content of the obtained acrylic adhesive, Denacor EX-212 as a curing agent was added in an amount of 0.5 parts by weight in terms of nonvolatile content, and the mixture was stirred to obtain a coating solution. An adhesive tape was obtained in the same manner as in Example 1.

### Comparative Example 5

5.5 parts of butyl acrylate, 10 parts of lauryl acrylate, 30 parts of methoxyethyl acrylate, 3.5 parts of 2-hydroxyethyl acrylate, 1.0 part of acrylamide, 75 parts of methyl ethyl ketone as a polymerization solvent, and 0.2 parts of azobisisobutyronitrile were charged into a four-necked flask equipped with a stirrer, a reflux condenser, a nitrogen inlet tube, a thermometer and a dropping funnel, and the temperature was raised to 80° C. while nitrogen gas was introduced. 5.5 parts of butyl acrylate, 10 parts of lauryl acrylate, 30 parts of methoxyethyl acrylate, 3.5 parts of 2-hydroxyethyl acrylate, 1.0 part of acrylamide, 75 parts of methyl ethyl ketone, and 0.2 parts of azobisisobutyronitrile as an initiator were charged into the dropping funnel, the mixture was added dropwise, and the polymerization reaction was carried out at 80° C. under a nitrogen atmosphere for 7 hours. After completion of the reaction, dilution was performed by adding 36 parts of ethyl acetate to obtain an adhesive having a nonvolatile content of 35% and a weight average molecular weight of 300,000.

<Preparation of Coating Solution>

With respect to 100 parts of the nonvolatile content of the obtained acrylic adhesive, Takenate D-110N as a curing agent was added in an amount of 0.4 parts by weight in terms of nonvolatile content, and the mixture was stirred to obtain a coating solution. An adhesive tape was obtained in the same manner as in Example 1.

### Comparative Example 6

5.5 parts of butyl acrylate, 10 parts of lauryl acrylate, 30 parts of methoxyethyl acrylate, 3.5 parts of 2-hydroxyethyl acrylate, 1.0 part of acrylamide, 50 parts of ethyl acetate as a polymerization solvent, and 0.2 parts of azobisisobutyronitrile were charged into a four-necked flask equipped with a stirrer, a reflux condenser, a nitrogen inlet tube, a thermometer and a dropping funnel, and the temperature was raised to 80° C. while nitrogen gas was introduced. 5.5 parts of butyl acrylate, 10 parts of lauryl acrylate, 30 parts of methoxyethyl acrylate, 3.5 parts of 2-hydroxyethyl acrylate, 1.0 part of acrylamide, 50 parts of ethyl acetate, and 0.2 parts of azobisisobutyronitrile as an initiator were charged into the dropping funnel, the mixture was added dropwise, and the polymerization reaction was carried out at 80° C. under a nitrogen atmosphere for 7 hours. After completion of the reaction, dilution was performed by adding 86 parts of ethyl acetate to obtain an adhesive having a nonvolatile content of 35% and a weight average molecular weight of 900,000.

<Preparation of Coating Solution>

With respect to 100 parts of the nonvolatile content of the obtained acrylic adhesive, Takenate D-110N as a curing agent was added in an amount of 0.4 parts by weight in terms of nonvolatile content, and the mixture was stirred to obtain a coating solution. An adhesive tape was obtained in the same manner as in Example 1.

TABLE 1

| | | Ex. | Ex. | Ex. | Ex. | Ex. | Ex. | Ex. | Ex. | Ex. | Ex. | Ex. | Ex. | Ex. | Ex. | Ex. | Ex. | Ex. |
|---|---|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 | 16 | 17 |
| (a-1) | BA | | | | | | 5.0 | | | | | | | | | | | |
| | 2HEA | 52.0 | 50.0 | 50.0 | | 12.0 | 12.0 | 8.0 | 8.0 | 38.0 | 9.5 | 9.5 | 7.5 | 14.0 | 13.0 | 13.0 | 13.0 | 16.7 |
| | IOA | | | | | | | | | | | | | | | | | |
| | INA | | | | | | | | | | | | | | | | | |
| | IDMA | | | | | | | | | | | | | | | | | |
| | LA | | | | | | | | | | | | | | | | | |
| | LMA | | | | 50.0 | | 2.0 | 30.0 | 20.0 | 5.0 | 18.0 | 18.0 | 18.0 | 15.0 | 15.0 | 15.0 | 15.0 | 12.0 |
| | ISA | | | | | | | | | | | | | | | | | |
| (a-2) | 2HEA | 5.0 | 5.0 | 5.0 | 5.0 | 5.0 | 5.0 | | | | | | | | | | | |
| | 4HBA | | | | | | | 1.0 | 1.0 | 1.0 | 1.0 | 1.0 | 1.0 | 1.0 | 0.5 | 0.5 | 0.5 | 0.3 |
| | 55PET-800 | | | | | | | | | | | | | | | | | |
| | PKA-5001 | | | | | | | | | | | | | | | | | |
| (a-3) | MEA | 40.0 | 30.0 | 40.0 | 30.0 | 50.0 | 50.0 | 50.0 | 50.0 | 50.0 | | | | | 60.0 | 60.0 | 60.0 | 60.0 |
| | MEMA | | 10.0 | | | | | | | | | | | | | | | |
| | EEA | | | | | | | | | | 55.0 | 55.0 | 55.0 | 55.0 | | | | |
| (a-4) | EC-A | | | | | 32.0 | 25.0 | 10.0 | 20.0 | 5.0 | 15.0 | 15.0 | 15.0 | 15.0 | | | | |
| | MTG-A | | | | | | | | | | | | | | 10.0 | | | |
| | DPM-A | | | | | | | | | | | | | | | 10.0 | | |
| | M-90G | | | | | | | | | | | | | | | | 10.0 | |
| | AM-900 | | | | | | | | | | | | | | | | | 10.0 |
| | MPE400A | | | | | | | | | | | | | | | | | |
| | BC | | | | | | | | | | | | | | | | | |
| | 041MA | 3.0 | 5.0 | 5.0 | 5.0 | | | | | | | | | | | | | |
| | PKA-5008 | | | | | | | | | | | | | | | | | |
| | PKA-5015 | | | | | | | | | | | | | | | | | |
| Other | AA | | | | | | | | | | | | 2.0 | | | | | |
| monomers | AM | | | | | 1.0 | 1.0 | 1.0 | 1.0 | 1.0 | | | | | | | | |
| | NVP | | | | | | | | | | 1.5 | 1.5 | 1.5 | | 1.5 | 1.5 | 1.5 | |
| | ACMO | | | | | | | | | | | | | | | | | 1.0 |
| Weight average | | 80 | 90 | 90 | 120 | 90 | 110 | 80 | 80 | 100 | 100 | 40 | 100 | 100 | 120 | 120 | 120 | 120 |
| molecular weight | | | | | | | | | | | | | | | | | | |
| Solubility | | ◯ | ◯ | ◯ | ◯ | ◯ | ◯ | ◯ | ◯ | ◯ | ◯ | Δ | ◯ | ◯ | ◯ | ◯ | ◯ | ◯ |
| Curing | HT | 2.0 | 2.0 | 9.0 | 2.0 | | | | | | | | | | | | | |
| agent | N3200 | | | | | 1.0 | 1.0 | 1.0 | 1.0 | 1.0 | | | | | | | | |
| | N3300 | | | | | | | | | | 1.0 | 1.0 | 1.0 | 1.0 | | | | |
| | L | | | | | | | | | | | | | | 0.5 | 0.5 | 0.5 | 0.3 |
| | 2030 | | | | | | | | | | | | | | | | | |
| | D-110N | | | | | | | | | | | | | | | | | |
| | EX-212 | | | | | | | | | | | | | | | | | |
| Moisture permeability | | Δ | Δ | Δ | Δ | ⊚ | ⊚ | ◯ | ⊚ | Δ | ⊚ | ⊚ | ⊚ | ⊚ | ⊚ | ⊚ | ⊚ | ⊚ |
| Adhesive | Adhesive | ◯ | ◯ | ◯ | ◯ | ◯ | ◯ | ◯ | ◯ | ◯ | ◯ | ◯ | ◯ | ◯ | ◯ | ◯ | ◯ | ◯ |
| strength | strength | | | | | | | | | | | | | | | | | |
| | (1) | | | | | | | | | | | | | | | | | |
| | Adhesive | ◯ | ◯ | ◯ | ◯ | ◯ | ◯ | ◯ | ◯ | ◯ | ◯ | ◯ | ◯ | ◯ | ◯ | ◯ | ◯ | ◯ |
| | strength | | | | | | | | | | | | | | | | | |
| | (2) | | | | | | | | | | | | | | | | | |
| | Adhesive | ◯ | ◯ | ◯ | ◯ | ◯ | ◯ | ◯ | ◯ | ◯ | ◯ | ◯ | ◯ | ◯ | ◯ | ◯ | ◯ | ◯ |
| | strength | | | | | | | | | | | | | | | | | |
| | ratio | | | | | | | | | | | | | | | | | |

| | | | | | | | | | | | | | | | | | |
|---|---|---|---|---|---|---|---|---|---|---|---|---|---|---|---|---|---|
| Adhesion to skin | Δ | Δ | Δ | A | Δ | Δ | Δ | ◯ | ◯ | ◯ | Δ | ◯ | ◯ | ◯ | ◯ | ◯ | ◯ |
| Irritation to skin-1 | Δ | Δ | Δ | Δ | Δ | ◯ | ◯ | ◯ | ◯ | ◯ | Δ | Δ | Δ | ◯ | ◯ | ◯ | ◯ |
| Irritation to skin-2 | ◯ | ◯ | ◯ | ◯ | ◯ | ◯ | ◯ | ◯ | ◯ | ◯ | ◯ | Δ | ◯ | ◯ | ◯ | ◯ | ◯ |

TABLE 2

| | | | | | | | | | | | | | Com- | Com- | Com- | Com- | Com- | Com- |
|---|---|---|---|---|---|---|---|---|---|---|---|---|---|---|---|---|---|---|
| | | | | | | | | | | | | | par- | par- | par- | par- | par- | par- |
| | | Ex. | Ex. | Ex. | Ex. | Ex. | Ex. | Ex. | Ex. | Ex. | Ex. | Ex. | ative | ative | ative | ative | ative | ative |
| | | 18 | 19 | 20 | 21 | 22 | 23 | 24 | 25 | 26 | 27 | 28 | Ex. 1 | Ex. 2 | Ex. 3 | Ex. 4 | Ex. 5 | Ex. 6 |
| (a-1) | BA | | | | | | | | | 5.0 | | | | | | | 11.0 | 11.0 |
| | 2HEA | 16.7 | 17.0 | 5.0 | 20.0 | 19.0 | 20.0 | 9.7 | 9.7 | 10.0 | 10.5 | 2.5 | 83.0 | | | 53.0 | | |
| | IOA | | | | | | | | | | | | | 37.0 | | | | |
| | INA | | | | | | | | | | | | | | 85.0 | | | |
| | IDMA | | | | | | | 8.0 | 8.0 | | | | | | | | | |
| | LA | | | | | | | | | | | | | | | | 20.0 | 20.0 |
| | LMA | 12.0 | 12.0 | 12.0 | | | | | | 3.0 | | | | | | | | |
| | ISA | | | | 5.0 | 5.0 | 5.0 | | | | 3.0 | 1.0 | 10.0 | | | | | |
| (a-2) | 2HEA | | | | | | | 0.3 | 0.3 | 1.0 | 1.0 | 1.0 | 5.0 | | | | 7.0 | 7.0 |
| | 4HBA | 0.3 | 0.05 | 12.0 | 1.5 | 1.5 | 1.5 | | | | | | | | | | | |
| | 55PET-800 | | | | | | | 5.0 | | | | | | | | | | |
| | PKA-5001 | | | | | | | | 5.0 | | | | | | | | | |
| (a-3) | MEA | 60.0 | 60.0 | 60.0 | | | | 70.0 | 70.0 | 75.0 | | | | | 30.0 | 45.0 | 60.0 | 60.0 |
| | EEA | | | | 65.0 | 65.0 | 65.0 | | | | 80.0 | 80.0 | | | | | | |
| (a-4) | EC-A | | | | 8.0 | 8.0 | | | | | | | | | | | | |
| | MTG-A | | | | | | | | | 5.0 | 5.0 | 15.0 | | | | | | |
| | DPM-A | | | | | | | | | | | | | | | | | |
| | M-90G | | | | | | | | | | | | | | | | | |
| | AM-90G | | | | | | | | | | | | | 60.0 | | | | |
| | MPE400A | 10.0 | 10.0 | 10.0 | | | | | | | | | | | | | | |
| | BC | | | | | | 8.0 | | | | | | | | | | | |
| | 041MA | | | | | | | | | | | | | | | | | |
| | PKA-5008 | | | | | | | 5.0 | | | | | | | | | | |
| | PKA-5015 | | | | | | | | 5.0 | | | | | | | | | |
| Other | AA | | | | | 1.0 | | | | | | | | | 5.0 | 2.0 | | |
| monomers | AM | | | | | | | | | | | | | | | | 2.0 | 2.0 |
| | NVP | | | | 0.5 | 0.5 | 0.5 | 2.0 | 2.0 | 1.0 | 0.5 | 0.5 | 2.0 | | | | | |
| | ACMO | 1.0 | 1.0 | 1.0 | | | | | | | | | | 3.0 | | | | |
| Weight average molecular | | 120 | 100 | 120 | 80 | 80 | 80 | 60 | 60 | 80 | 100 | 100 | 70 | 60 | 80 | 90 | 30 | 90 |
| weight | | | | | | | | | | | | | | | | | | |
| Solubility | | ◯ | ◯ | ◯ | ◯ | ◯ | ◯ | ◯ | ◯ | ◯ | Δ | Δ | ◯ | Δ | ◯ | ◯ | ◯ | ◯ |
| Curing | HT | | | | | | | | 0.5 | 0.5 | 0.5 | | 0.1 | | | | | |
| agent | N3200 | | | | | | | | | | | | | | | | | |
| | N3300 | | | | | | | | | | | | | | | | | |
| | L | 0.3 | 0.3 | 0.3 | | | | | | | | 1.0 | | | | | | |
| | 2030 | | | | 1.0 | 1.0 | 1.0 | | | | | | | | | | | |
| | D-110N | | | | | | | 1.0 | 1.0 | | | | | | | | 0.4 | 0.4 |
| | EX-212 | | | | | | | | | | | | | | | 0.5 | | |
| Moisture permeability | | ⊚ | ⊚ | ⊚ | ⊚ | ⊚ | ⊚ | ◯ | ◯ | ◯ | ⊚ | ⊚ | X | ◯ | Δ | Δ | ◯ | ◯ |
| Adhesive | Adhesive | ◯ | ◯ | ◯ | ◯ | ◯ | ◯ | ◯ | ◯ | ◯ | ◯ | X | X | X | ◯ | ◯ | ◯ | ◯ |
| strength | strength (1) | | | | | | | | | | | | | | | | | |
| | Adhesive | ◯ | ◯ | ◯ | ◯ | ◯ | ◯ | ◯ | ◯ | ◯ | ◯ | ◯ | ◯ | X | ◯ | ◯ | ◯ | ◯ |
| | strength (2) | | | | | | | | | | | | | | | | | |
| | Adhesive | ◯ | ◯ | ◯ | ◯ | ◯ | ◯ | ◯ | ◯ | ◯ | ◯ | ◯ | X | X | ◯ | ◯ | ◯ | ◯ |
| | strength | | | | | | | | | | | | | | | | | |
| | ratio | | | | | | | | | | | | | | | | | |

| | | | | | | | | | | | | | | | | | |
|---|---|---|---|---|---|---|---|---|---|---|---|---|---|---|---|---|---|
| Adhesion to skin | ◯ | ◯ | Δ | ◯ | ◯ | ◯ | ◯ | ◯ | ◯ | Δ | Δ | X | ◯ | X | X | X | X |
| Irritation to skin-1 | ◯ | Δ | Δ | ◯ | Δ | ◯ | Δ | Δ | Δ | Δ | Δ | X | Δ | X | X | X | X |
| Irritation to skin-2 | ◯ | Δ | Δ | ◯ | Δ | ◯ | Δ | Δ | ◯ | ◯ | ◯ | X | X | X | X | Δ | Δ |

1. An adhesive, comprising a copolymer which at least comprises monomer units represented by (a-1), (a-2), (a-3), and (a-4) described below, and the following content is contained among all of the monomers which constitute the copolymer:
(a-1) 5 to 45 wt % of an alkyl (meth)acrylate;
(a-2) 0.01 to 10 wt % of a (meth)acrylate having a hydroxyl group or an allyl ether having a hydroxyl group;
(a-3) 50 to 78 wt % of an alkoxyalkyl (meth)acrylate;
(a-4) 1 to 25 wt % of an alkoxypolyalkylene glycol mono(meth)acrylate or an alkoxypolyalkylene glycol allyl ether.

2. The adhesive according to claim 1, wherein a solubility in water at 40° C. is less than 3%.

3. The adhesive according to claim 1, wherein the copolymer has a weight average molecular weight of 800,000 or more and less than 2,000,000.

4. The adhesive according to claim 1, further comprising a curing agent.

5. The adhesive according to claim 1, comprising a copolymer which at least includes the monomer units represented by (a-1), (a-2), (a-3), and (a-4), and a monomer unit having an amide bond.

6. The adhesive according to claim 1, wherein the copolymer is a copolymer that do not contain monomers having an acidic group.

7. The adhesive according to claim 1, wherein the alkyl (meth)acrylate (a-1) comprises an alkyl (meth)acrylate having 4 to 8 carbon atoms at 5 to 40 wt % and an alkyl (meth)acrylate having 9 to 22 carbon atoms at 5 to 40 wt %.

8. The adhesive according to claim 1, wherein the adhesive is used for medical applications.

9. The adhesive according to claim 1, wherein, after an adhesive tape having an adhesive layer formed of the adhesive according to claim 1 is pressure-bonded to a stainless steel plate in an environment of 23° C. and 50% RH, when an adhesive strength measured after 10 minutes in an environment of 23° C. and 50% RH is defined as adhesive strength (1) and an adhesive strength measured after 24 hours in an environment of 40° C. and 50% RH is defined as adhesive strength (2), a ratio of adhesive strength (2)/adhesive strength (1) is less than 3.

10. A method of preparing an adhesive, in which monomers represented by (a-1), (a-2), (a-3), and (a-4) described below are copolymerized, and which is characterized by copolymerizing the monomers having the following contents among all of the monomers which constitute a copolymer:
(a-1) 5 to 45 wt % of an alkyl (meth)acrylate;
(a-2) 0.01 to 10 wt % of a (meth)acrylate having a hydroxyl group or an allyl ether having a hydroxyl group;
(a-3) 50 to 78 wt % of an alkoxyalkyl (meth)acrylate;
(a-4) 1 to 25 wt % of an alkoxypolyalkylene glycol mono(meth)acrylate or an alkoxypolyalkylene glycol allyl ether.

11. An adhesive tape, comprising an adhesive layer formed of the adhesive according to claim 1.

12. The adhesive tape according to claim 11, wherein, after the adhesive tape having the adhesive layer formed of the adhesive is pressure-bonded to a stainless steel plate in an environment of 23° C. and 50% RH, when an adhesive strength measured after 10 minutes in an environment of 23° C. and 50% RH is defined as adhesive strength (1) and an adhesive strength measured after 24 hours in an environment of 40° C. and 50% RH is defined as adhesive strength (2), a ratio of adhesive strength (2)/adhesive strength (1) is less than 3.

13. A method of preparing an adhesive tape, in which the adhesive according to claim 1 is applied onto a substrate.

14. The adhesive according to claim 2, wherein the copolymer has a weight average molecular weight of 800,000 or more and less than 2,000,000.

15. The adhesive according to claim 2, further comprising a curing agent.

16. The adhesive according to claim 3, further comprising a curing agent.

17. The adhesive according to claim 2, comprising a copolymer which at least includes the monomer units represented by (a-1), (a-2), (a-3), and (a-4), and a monomer unit having an amide bond.

18. The adhesive according to claim 3, comprising a copolymer which at least includes the monomer units represented by (a-1), (a-2), (a-3), and (a-4), and a monomer unit having an amide bond.

19. The adhesive according to claim 4, comprising a copolymer which at least includes the monomer units represented by (a-1), (a-2), (a-3), and (a-4), and a monomer unit having an amide bond.

20. The adhesive according to claim 2, wherein the copolymer is a copolymer that do not contain monomers having an acidic group.
